(19) United States
(12) Patent Application Publication
(10) Pub. No.: US 2017/0027559 A1
(43) Pub. Date: Feb. 2, 2017

(54) System and Method for Soft Tissue Repair

(71) Applicants: David J. Schneider, Lafayette, CO (US); Eric P. Guyer, Johns Creek, GA (US)

(72) Inventors: David J. Schneider, Lafayette, CO (US); Eric P. Guyer, Johns Creek, GA (US)

(21) Appl. No.: 15/221,360

(22) Filed: Jul. 27, 2016

Related U.S. Application Data
(60) Provisional application No. 62/197,308, filed on Jul. 27, 2015.

(51) Int. Cl. A61B 17/04

(52) U.S. Cl. CPC A61B 17/0483; A61B 17/0401; A61B 2017/047; A61B 2017/0409; A61B 2017/00022

(57) ABSTRACT

A system and method for soft tissue repair. According to one embodiment, sensing gauges or other sensing technologies are utilized at distal ends of jaws, passing a needle or within the instrument to identify tissue differences that give the end user feedback on these differences. A console connected to the instrument assists in utilizing sensing technology to give the end user feedback on tissue differences.

### CROSS REFERENCE TO RELATED APPLICATIONS

The present application claims the benefit of priority to U.S. Provisional Patent Application Ser. No. 62/197,308 titled “System and Method for Soft Tissue Repair” filed Jul. 27, 2015, the contents of which are hereby incorporated by reference in their entirety.

### FIELD

The present application relates generally to medical devices and procedures and more specifically to systems and methods for soft tissue repairs, such as treating rotor cuff repairs and other soft tissue pathologies.

### BACKGROUND

Rotator cuff tears are common in patients of all ages and athletic condition. Rotator cuff tears are treated with a variety of shoulder anchor geometries, materials and suture configurations. In some cases, a suture is passed through diseased or torn tissue in order to complete the surgical repair. The repair is completed under direct visualization (‘open’ or ‘mini-open’) or with the use of arthroscopic visualization equipment. In all of these techniques, the use of a suture-passing device is required to pass suture through the soft tissue.

Failure rates in rotator cuff repair are relatively high for a number of biological and mechanical reasons. Poor or diseased rotator cuff tissue quality makes it difficult for a surgeon to adequately secure a suture into the rotator cuff. Because the rotator cuff is under tension both during and after the repair, there is significant load on the anchor or fixation point and the suture that was passed through the rotator cuff during the repair. Therefore, it is difficult to ensure that the sutures used to secure the rotator cuff repair can endure the load during the surgery and for a period of healing time following the procedure.

It is within the aforementioned context that a need for the present invention has arisen. Thus, there is a need to address one or more of the foregoing disadvantages of conventional systems and methods, and the present invention meets this need.

### SUMMARY

Various aspects of systems and methods for soft tissue repair can be found in exemplary embodiments of the present invention.

According to one embodiment, sensing gauges or other sensing technologies are utilized at distal ends of jaws, passing a needle or within the instrument to identify tissue differences that give the end user feedback on these differences. A console connected to the instrument assists in utilizing sensing technology to give the end user feedback on tissue differences.

Formulations and methods of the present invention are applied to the treatment of a variety of surgical indications and soft tissue abnormalities. The present methods and systems are utilized in the surgical treatment of rotator cuff tears or other soft tissue pathologies that would benefit from better understanding tissue quality during a procedure (e.g., identifying glenohumeral ligaments). A suture passed through such tissue (likely higher strength, greater density or integrity) will have a greater likelihood of success compared to a suture passed through poorer quality tissue.

A further understanding of the nature and advantages of the present invention herein may be realized by reference to the remaining portions of the specification and the attached drawings. Further features and advantages of the present invention, as well as the structure and operation of various embodiments of the present invention, are described in detail below with respect to the accompanying drawings. In the drawings, the same reference numbers indicate identical or functionally similar elements.

### BRIEF DESCRIPTION OF THE DRAWINGS

FIGS. 1A-1C illustrate sensing technologies in accordance with an exemplary embodiment of the present invention.

FIGS. 2A-2C illustrate sensing technologies in accordance with an exemplary embodiment of the present invention.

FIG. 3 illustrates a suture passing needle in accordance with an exemplary embodiment of the present invention.

FIG. 4 illustrates a suture passing device in accordance with an exemplary embodiment of the present invention.

FIG. 5 illustrates a process for treating rotator cuff repairs and other soft tissue abnormalities in accordance with an exemplary embodiment of the present invention.

### DETAILED DESCRIPTION

Reference will now be made in detail to the embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth to provide a thorough understanding of the present invention. However, it will be obvious to one of ordinary skill in the art that the present invention may be practiced without these specific details. In other instances, well-known methods, procedures, components, and circuits have not been described in detail as to not unnecessarily obscure aspects of the present invention.

Arthroscopic, mini-open or open techniques allow a surgeon to visualize the rotator cuff tear during surgery and subsequently attempt to repair the torn tendon. Under direct visualization or with the use of an arthroscopic scope and camera, the surgeon can use different fixation products that allow him or her to reattach the torn tendon to bone. A shoulder anchor or some geometry and material are inserted into the humerus and sutures that are loaded into the anchor are passed through the rotator cuff. The sutures are tied down with different techniques and the anchor is designed to hold its position in the bony humerus. Various limitations associated with these techniques have been described above.

According to one embodiment, sensing gauges or other sensing technologies are utilized at the distal end of the jaws, in or on the suture passing needle or within the instrument to identify tissue differences that give the end user feedback on these differences. A console can be connected to the instrument assists in utilizing sensing technology to give the end user feedback on tissue differences.

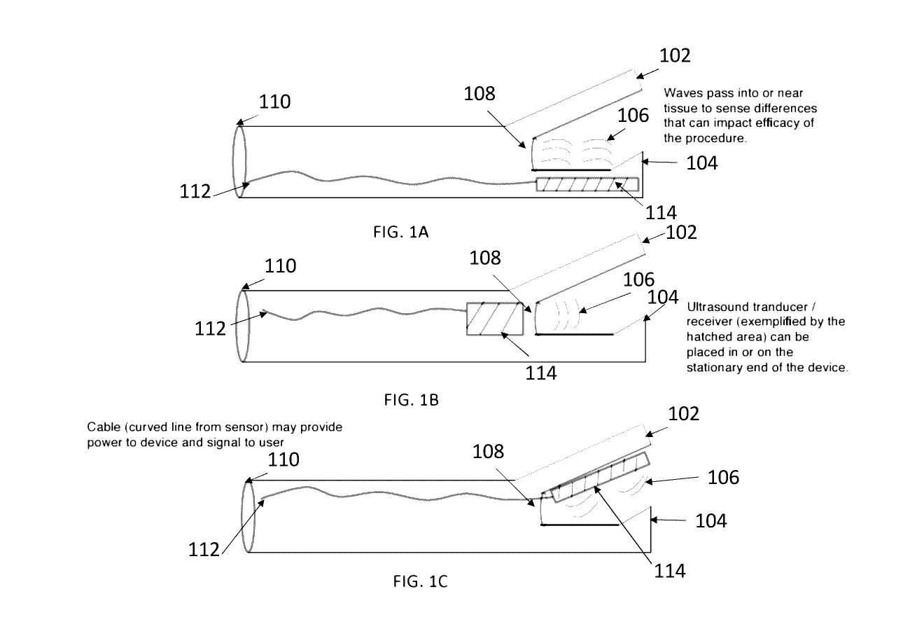

FIGS. 1A-1C illustrate sensing technologies in accordance with an exemplary embodiment of the present invention. Ultrasound device or devices are placed in, on, under or near the distal end of suture passing jaws to identify tissue differences. A handle is used to actuate the jaw or jaws when gasping tissue.

In FIGS. 1A-1C, an exemplary apparatus includes a shaft having a proximal end 110 and a distal end, the distal end including a distal top jaw tip 102 that is a movable tissue clamp. The distal end further includes a distal bottom jaw tip 104 that is a bottom tissue clamp, being either fixed or moveable. In the distal tip, there is a pivot point 108 for the tissue clamps 102, 104. The apparatus shaft carries signal and power cables 112, and a suture passing needle and suture (not shown). An ultrasonic device 114 can be placed in or on a stationary end of the apparatus (e.g., in the bottom clamp, the top clamp, near the pivot point 108). It will be appreciated that ultrasonic device 114 can be one or more ultrasonic devices or other devices utilizing other sensor technology.

Waves 106 pass into or near the tissue to sense differences that can impact efficacy of the procedure. Waves are ultrasonic communication between a transducer and receiver.

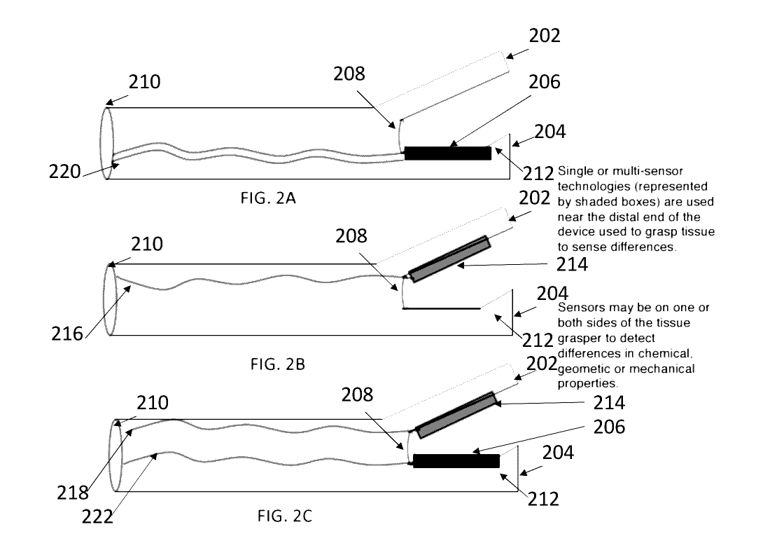

FIGS. 2A-2C illustrate sensing technologies in accordance with an exemplary embodiment of the present invention. Sensing technologies are placed in the distal end of suture passing jaws that is connected to signal transmission. The transmission may be connected or able to communicate to a device that gives the end user feedback on the tissue differences to identify the best location for passing a suture.

In FIGS. 2A-2C, an exemplary apparatus includes a shaft having a proximal end 210 and a distal end, the distal end including a distal top jaw tip 202 that is a movable tissue clamp. The distal end further includes a distal bottom jaw tip 204 that is a bottom tissue clamp, being either fixed or moveable. In the distal tip, there is a pivot point 208 for the tissue clamps 202, 204. The apparatus shaft carries signal and power cables 216, 218, 220, 222 (according to the design shown), and a suture passing needle and suture (not shown). As illustrated in FIGS. 2A-2C, signal and power cables can be mounted on the upper tissue clamp, on the bottom tissue clamp, or a combination of the two. An ultrasonic device 206, 214 can be placed in or on a stationary end of the apparatus (e.g., in the bottom clamp, the top clamp, or a combination of the two). It will be appreciated that ultrasonic devices 206, 214 can be one or more ultrasonic devices or devices utilizing other sensor technology.

Upper and lower tissue clamps grasp tissue and sense differences relevant to the procedure taking place. Sensors detect differences in chemical, geometric, or mechanical properties of the tissue being grasped.

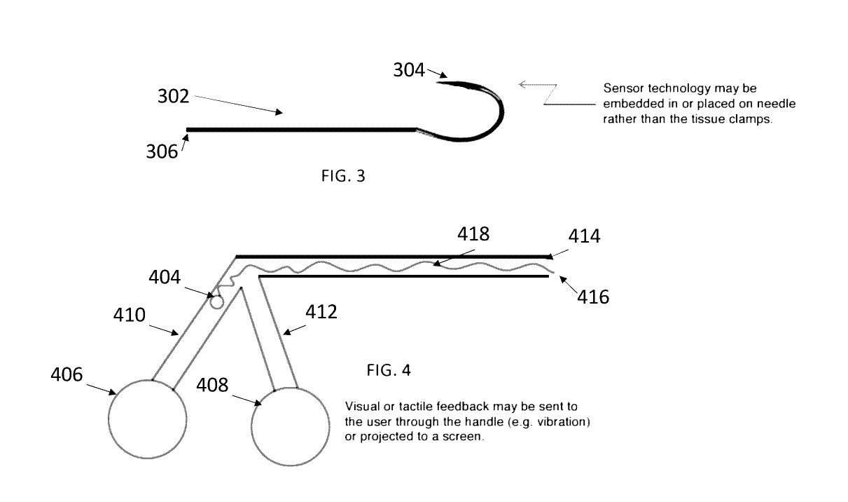

FIG. 3 illustrates a suture passing needle in accordance with an exemplary embodiment of the present invention. The suture passing needle can be utilized with any of the embodiments described herein.

In FIG. 3, a suture passing needle shaft 302 has a proximal end 306 and a tip 304 at a distal end. The tip 304 includes sensing technology embedded in the needle or placed on the needle for localized probing of tissue properties.

FIG. 4 illustrates a suture passing device in accordance with an exemplary embodiment of the present invention. Suture passing device does not utilize jaws. This embodiment illustrates the ability to place the sensing technologies in any mechanical suture passing device.

In FIG. 4, an apparatus includes a device shaft having a distal tip 414 and device handles 410, 412 attached to a proximal end of the device shaft. One or more of device handles 410 and 412 can be fixed, while the other is moveable. Device handles 410, 412 include grips 406 and 408. The apparatus includes a signal transducer, receiver, or other indicator 404 for sensing differences in tissue characteristics inside the shaft near the distal end of the shaft. The apparatus further includes a sensing or power cable 418, having a distal end 416 near the distal tip 414 of the shaft. Visual or tactile feedback may be sent to the user through the handles (e.g., vibration) or projected to a display.

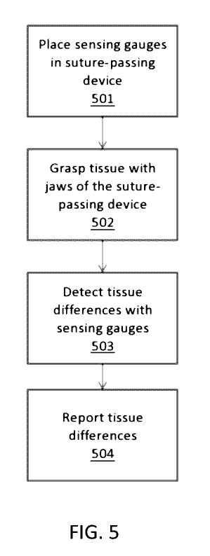

FIG. 5 illustrates a process for treating rotator cuff repairs and other soft tissue abnormalities in accordance with an exemplary embodiment of the present invention.

The process includes using sensing gauges or other technologies to identify structurally stronger, denser, or any differences in soft tissue that assist in determining where to selectively pass sutures and place implants. Such sensing technologies are placed in a suture-passing device 501. The sensing technologies or gauges can be placed in the distal ends of the mechanical jaw of a suture-passing device. These gauges are placed such that when tissue is grasped by the jaws of the passer, the sensing gauges are on opposing sides of each other with tissue between the gauges. These sensing gauges are alternatively placed in the passing needle or wire. In addition, the gauges can be placed anywhere within the passing instrument or a connected console such that it can determine differences in soft tissue. When placed in the distal jaws, the gauges detect tissue differences 503 as the end user articulates the jaws and grasps 502, touches or is in close proximity to different areas of tissue. The tissue differences can be reported 504. FIGS. 1A-2C illustrate how gauges can be oriented on the jaws and utilized in conjunctions with passing suture.

According to one embodiment, a method of surgically treating rotator cuff tears utilizes sensing technology like pressure, strain, stress, capacitance, density, acoustic, biological, optical, fluorescing, magnetic, electro-optical, electrochemical, ionizing, ultrasound, vibration, lab-on-chip and the like to identify tissue differences not available with current techniques. According to embodiments, the method of surgically treating rotator cuff tears is combined with a mechanical suture passing instrument to identify tissue differences.

According to one embodiment, a method comprises utilizing ultrasound in combination with a mechanical suture passing instrument to identify tissue difference, such that ultrasound is housed within the suture passing device.

According to one embodiment, a method comprises identifying striation patterns, tissue density, tissue strength, thickness, or quality otherwise and allowing a surgeon to optimize suture and/or anchor placement when passing sutures with suture passing instruments.

According to one embodiment, a device utilizes distal ends of a jaw of a suture passing instrument to place the strain gauges or other sensing technologies to determine and indicate differences in soft tissue.

According to one embodiment, a device utilizes a suture passing needle to place sensing technologies to indicate soft tissue differences.

According to one embodiment, a device utilizes a distal tip of a suture passing device to place sensing gauges or other technologies that indicate differences in tissue.

According to one embodiment, a device utilizes technology like said sensing technologies disclosed herein in conjunction with a passing suture to identify differences in tissue quality.

According to one embodiment, a device provides visual, tactile or audible feedback to an end user to indicate soft tissue differences when passing sutures or placing implant.

According to one embodiment, a device translates information from a gauge or other sensing technology contained in the suture-passing instrument to a visual indicator on the screen during an arthroscopy such that a surgeon can visualize tissue differences and inhomogeneity's prior to passing suture. The visual queue allows the surgeon to better identify the optimal location to place sutures and or implants.

According to one embodiment, a device includes a console or like device is connected to the suture passing instrument with sensing technology, whereas the console assists in interpreting the feedback from the differences in tissue quality.

According to one embodiment, a device includes a console connected via cable, Bluetooth or other tethered or wireless methods to interpret the differences in soft tissue detected from the sensing technology within the suture passing device.

According to one embodiment, a device includes sensing technology contained within the suture passing instrument and it communicates or is paired with a display that gives the end user feedback on the tissue differences at the operative site where suture is intended on being passed.

According to one embodiment, a method includes placing ultrasound technology within the suture-passing instrument to identify differences in soft tissue while passing suture.

According to one embodiment, a method of identifying tissue differences utilizes electrical, chemical, mechanical, optical, geometric or other physical property differences (inhomogeneity's) in conjunction with all suture passing instruments used to treat rotator cuff repairs and other operative soft tissue repairs where tissue quality plays a role in repair technique and where one may pass sutures, place implants or resect tissue.

While the above is a complete description of exemplary specific embodiments of the invention, additional embodiments are also possible. Thus, the above description should not be taken as limiting the scope of the invention, which is defined by the appended claims along with their full scope of equivalents.

We claim:

1. A suture passing instrument, comprising:
a shaft having a proximal end and a distal end, wherein the distal end has a distal top jaw tip that is a first tissue clamp and a distal bottom jaw tip that is a second tissue clamp,
a pivot point attached to the shaft positioned between the distal top jaw tip and the distal bottom jaw tip,
a signal and power cable extending from the proximal end of the shaft to the distal end of the shaft,
a suture passing needle and suture, and
a sensing device positioned in one or more of the first tissue clamp, the second tissue clamp, and the pivot point,
wherein the sensing device collects and provides data regarding tissue clamped between the first tissue clamp and the second tissue clamp such that the data regarding the tissue can be used to select a location for placement of the suture.

2. The suture passing instrument of claim 1, wherein the distal bottom tip is one of fixed or movable.

3. The suture passing instrument of claim 1, wherein the signal and power cable are included in a single cable.

4. The suture passing instrument of claim 1, wherein the signal and power cables are separate and distinct cables.

5. The suture passing instrument of claim 1, wherein the sensing device is ultrasonic.

6. The suture passing instrument of claim 1, wherein the sensing device detects one or more of pressure, strain, stress, capacitance, density, acoustic, biological, optical, fluorescing, magnetic, electro-optical, electrochemical, ionizing, ultrasound, vibration, and lab-on-chip to identify differences in tissue that is clamped.

7. The suture passing instrument of claim 1, further comprising a communication module that transmits sensing data to a console for visual display of the sensing data.

8. The suture passing instrument of claim 7, wherein the communication module enables one or more of wired and wireless communication.

9. The suture passing instrument of claim 8, wherein the wireless communication is one or more of Bluetooth, WiFi, NFC, and cellular.

10. The suture passing instrument of claim 1, wherein the instrument provides a tactile feedback signal to the user based upon a condition of the tissue clamped.

11. The suture passing instrument of claim 10, wherein the condition is predetermined.

12. A method of optimizing suture and/or anchor placement in a surgery, comprising:
identifying one or more of striation patterns, tissue density, tissue strength, tissue thickness, and tissue quality, wherein the identifying is achieved through the use of a suture passing instrument according to claim 6, and
modifying placement of the suture and/or anchor, using the suture passing instrument, based upon the identification.
